(19) United States
(12) Patent Application Publication
(10) Pub. No.: US 2024/0115468 A1
(43) Pub. Date: Apr. 11, 2024

(54) COMPOSITION FOR TEETH DESENSITIZATION

(71) Applicant: ORAL SCIENCE INC., Brossard (CA)

(72) Inventor: Charles F. COX, Fenton, MI (US)

(21) Appl. No.: 18/481,429

(22) Filed: Oct. 5, 2023

Related U.S. Application Data
(60) Provisional application No. 63/413,348, filed on Oct. 5, 2022.

(51) Int. Cl. A61K 8/362; A61K 8/04; A61K 8/21; A61K 8/43; A61Q 11/00

(52) U.S. Cl. CPC A61K 8/362; A61K 8/044; A61K 8/21; A61K 8/43; A61Q 11/00; A61K 2800/82

(57) ABSTRACT

There is provided a composition for teeth desensitization. The composition comprises from 1 to 15 wt. % of suspended crystals of oxalic acid potassium salt dihydrate having a size of from 50 nm to less than 1000 nm. The composition comprises from 0.5 to 10 g/L of NaOH and has a pH of less than 2.

### CROSS-REFERENCE TO A RELATED APPLICATION

This disclosure claims priority from U.S. provisional application 63/413,348 filed on Oct. 5, 2022 and incorporated herein by reference in its entirety.

### TECHNICAL FIELD

This disclosure relates to the field of tooth pain and hypersensitivity, compositions and methods of treating same or reducing the symptoms thereof.

### BACKGROUND OF THE ART

Teeth desensitizing agents aim to relieve oral pain and reduce teeth sensitivity. Historically, one of the first clinical desensitizing agents was a copal resin varnish in a solvent such as chloroform or ether. Since then there has been a shift away from such resins and currently there are only a few commercial varnishes that use copal or amber resins. Other resins have emerged based on the initial resins identified. However, in general, resins are hazardous and present a health risk. Thus, they are not the preferred treatment option. Desensitizing agents that are based on aldehyde solutions, have also been developed as an alternative. For example, glutaraldehyde and formocresol have been used to treat tooth pain. Unfortunately, these aldehydes are also associated with toxicity. Earth metal agents such as calcium hydroxide Ca(OH)₂ have been used as liner pastes to provide relief from dentin sensitivity. However, their efficacy and action varies with different cavity depths in the dentin, particularly when there is more organic tissue towards the vital pulp and more mineral content towards the enamel-dentin junction (EDJ). Moreover, the liners are prone to surface cracking which provides a gateway of access for recurrent caries. Accordingly, improvements are desired for teeth desensitizing agents with reduced toxicity and improved efficacy.

### SUMMARY

There is provided a composition for teeth desensitization having reduced toxicity and improved efficacy. The composition includes from 1 to 15 wt. % of suspended crystals of oxalic acid potassium salt dihydrate having a size of from 50 nm to less than 1000 nm, and from 0.5 to 10 g/L of NaOH. The composition has a pH of less than 2.0. In some embodiments, the suspended crystals of oxalic acid potassium salt are present in a concentration of from 1.5 to 7 wt. %. In some embodiments, the concentration is of from 1.5 to 4 wt. %. In some embodiments, the size the suspended crystals of oxalic acid potassium salt is of from 100 to 800 nm. In some embodiments, the NaOH is present in a concentration of from 1 to 5 g/L. In some embodiments, a mass ratio of the oxalic acid potassium salt dihydrate crystals to the NaOH is from 5:1 to 15:1. In some embodiments, the ratio is from 8:1 to 12:1. In some embodiments, the pH is between 1 and 2 or between 1 and 1.9. In some embodiments, the composition further comprises fluoride. In some embodiments, the fluoride is present in a concentration of 100 ppm to 1500 ppm. In some embodiments, the composition further comprises high oxygen potential water. In some embodiments, the high oxygen potential water is present in a concentration of from 0.1 mL/L to 10 mL/L. In some embodiments, the composition further comprises a flavoring agent, a coloring agent, or an antiseptic agent. In some embodiments, the antiseptic agent is a bisbiguanide.

There is provided a method for producing a composition for teeth desensitization, the method including: mixing an oxalic acid potassium salt and a NaOH salt in an aqueous phase to obtain a mixture; heating the mixture to a temperature of from 28 to 40° C.; reducing the pH of the mixture to less than 2.0 to obtain an acidic mixture; and ultrasonicating the acidic mixture to obtain oxalic acid potassium dihydrate salt crystals having a size of less than 1000 nm. In some embodiments, the step of reducing the pH includes adding high oxygen potential water. In some embodiments, the step of ultrasonicating comprises ultrasonicating in a closed fluid circuit. In some embodiments, the step of heating is performed for 30 mins-1 h30. In some embodiments, the method further comprises after the step of reducing the pH and/or after the step of ultrasonicating, adding a fluoride.

Many further features and combinations thereof concerning the present improvements will appear to those skilled in the art following a reading of the instant disclosure.

### DESCRIPTION OF THE DRAWINGS

FIG. 1 is a scanning electron microscopy (SEM) image of a dentin floor that was treated with a control composition at a 2000× magnification.

FIG. 2 is a SEM image of a dentin floor treated with an exemplary composition at a 2000× magnification.

FIG. 3 is a SEM image of a dentin floor treated with an exemplary composition at a 5000× magnification.

FIG. 4 is a SEM image of a dentin floor treated with an exemplary composition at a 7000× magnification.

### DETAILED DESCRIPTION

There is provided an aqueous composition comprising oxalic acid potassium salt dihydrate crystals to treat tooth sensitivity, such as dental hypersensitivity, in humans and other mammals having a similar tooth structure of enamel, dentin, cementum, and periodontal anatomy and physiology. For example, the present composition is useful for sealing and desensitizing dentin as well as treating cementum defects. The composition generally comprises from 1 to 15 wt. %, with respect to a total weight of the composition, of oxalic acid potassium salt dihydrate crystals having a size of from 50 nm to less than 1000 nm. The size may be defined as the diameter of the crystals when the crystals have a spheroidal shape or in some cases where the crystals have an irregular shape, the size can be defined as the greatest distance between two opposite points at the surface of the crystals. The composition also comprises from 0.5 to 10 g/L of NaOH base which helps stabilize the size of the oxalic acid potassium salt dihydrate crystals in the nanometer range, for example in a size of less than 1000 nm, less than 900 nm, less than 800 nm, or less than 750 nm. The composition is an acidic composition that has a pH of less than 2 or less than 1.9, for example between 1 and 2, between 1 and 1.9, between 1 and 1.85 or between 1 and 1.8. The pH also plays a role in stabilizing the size of the oxalic acid potassium salt dihydrate crystals in the nanometer range.

The nanometer size range of the oxalic acid potassium salt dihydrate crystals is desirable for providing an improved teeth desensitization effect. The teeth desensitizing effect is achieved by reducing or stopping dentin tubule sensitivity with the oxalic acid potassium salt dihydrate crystals which are acid-resistant. More specifically, when the composition is delivered orally to a subject in need thereof, a chemical chelation reaction occurs between the oxalic acid potassium salt dihydrate crystals and calcium hydroxyapatite (HAp) found in the oral cavity to form a plurality of acid resistant calcium oxalate (CaOx) nanocrystals. In some embodiments, the plurality of acid resistant CaOX are present in a number in the order of 10⁴, 10⁵ or 10⁶ crystals (or more). In other words, millions of acid resistant calcium oxalate (CaOx) nanocrystals can be formed in the oral cavity when the present composition is administered to a subject in need thereof. The CaOx nanocrystals then block the hydrodynamic tubule fluid flow which causes tooth sensitivity and the CaOx nanocrystals prevent the physiological migration of cariogenic microorganisms into the dentin tubule complex towards the pulp. Oral pain is generally caused by an uncontrolled bidirectional hydraulic fluid flow in the dentin tubule complex-caused by any effect that opens the normally closed dentin tubule system of lateral canaliculi. The hydrodynamic fluid flow in the dentin tubule complex is also responsible, at least in part, for tooth sensitivity and caries complications. In some embodiments, the blocking of the hydrodynamic tubule is achieved by the increase in nanocrystal density driven by the chelation reaction. In some embodiments, the chelation reaction is a “rapid” reaction that can occur within 5 seconds of placement in an oral cavity. The chelation includes a chemical reaction with ionized calcium in the dentinal fluid forming an insoluble white precipitate of calcium oxalate that can occlude the dentinal tubules. This can lead to a decreased permeability of dentin, a decreased acid penetration of dentin and a decreased dentinal sensitivity.

The chelation of oxalic acid potassium salt dihydrate crystals form a plurality of CaOx nanocrystals that can serve as nano-liners of the operative oral cavity interface as well as to block the hydrodynamic fluid flow in the opened dentin tubules. The plurality of CaOX acid resistant nanocrystals can seal enamel lamella defects, as well as cracks and defects in the cavity floor. The CaOx nanocrystals can also occlude the dentin tubules as they are acid resistant. Indeed, the present composition is formulated at a pH of less than 2 and the oxalic acid potassium salt dihydrate crystals are stabilized in this pH range. Moreover, the present crystals in the nanometer range are smaller than traditional potassium salt crystals used for teeth desensitization. Accordingly, the oxalic acid potassium salt dihydrate crystals have an improved penetration and chelation when compared to other potassium salt desensitizers with a larger crystal size (e.g. in the micron range). The reduced size of the oxalic acid potassium salt dihydrate crystals can provide an improved diffusion in the oral cavity (for example into and around dentin tubules) which can then improve the speed and density at which the CaOx nanocrystals are formed, as well as the overall depth of crystals that may be formed. This can enable an improved sealing of the deeper affected enamel lamella and defects as well as a more rapid penetration into the dentin tubules due to a lower pH (e.g. less than 2, preferably equal or less than 1.85), which accelerates the chelation to the mineralized HAp tooth substrates. The improved diffusion also allows for an improved penetration into the EDJ interface region between the enamel and dentin complex in order to be able to seal the dentinal tubules against bacterial invasion as well as to swiftly minimize and quell the oral pain.

Enamel, dentin and cementum substrates of the tooth are each composed of various concentrations of HAp that are at an alkaline pH under physiological conditions. The remineralization of these substrates through the chelation of HAp may be considered as a similar process to the healing of soft-tissues. This biological process is not a regeneration of the substrate as there is no developmental cellular process to produce new tissue. The term remineralization is the reversal of the demineralization process with the deposition of inorganic crystals in the spaces that have been created by the HAp mineral loss, which can for example be due to caries activity. In these hard tissues, the minerals form crystal matrices that are seen as a crystalline appatite, usually a calcium phosphate.

The enamel surface of a human tooth along the gingival margin generally have many small pores that serve as diffusion channels through the amorphous enamel surface that is absent of any structural rods or deposited crystals. These pores permit the passage of organic bacterial acids, which are responsible for the formation of white spot lesions below the non-cavitated enamel surface. The blockage of such pores with the present composition can thus reduce or eliminate the formation of white spot lesions.

Generally dentin tubules have an irregular multiform morphology with non-planar surfaces. It is therefore an advantage to have nano sized oxalic acid potassium salt dihydrate crystals that can more easily penetrate the tubule to block fluid flow with chelation nanocrystals thanks to their size. A nanoparticle size can overcome the diffusion limitations caused by the irregular morphology of the dentin tubule compared to larger micron particle sizes. Moreover, the bonding to surfaces of enamel lamellae, and dentin tubules is also affected by the irregular topography of such surfaces. The nano sized crystals described herein can overcome bonding limitation and can more easily adhere to or come into contact with the irregular surfaces of the oral cavity. Moreover, a reduced particle size allows for a greater surface contact area and therefore an improved efficacy. Thus, the smaller the particle, the greater the chemical chelation ability to achieve greater surface contact with HAp in the oral cavity, for example the HAp of the enamel and dentin.

The present composition can achieve chemo-mechanical blockage of the dentin tubules even at their deepest and narrowest functional diameter, as well as for each enamel lamella, its cracks and defects. The oxalic acid potassium salt dihydrate crystals penetrate through the enamel lamella and its permeable channels to access the non-vital EDJ, which is an expansive thin but broad zone or gateway that permits microorganisms as well as the bidirectional hydrostatic flow of an interstitial fluid through the dentin tubules complex as well as cementum defects in the root dentin. The formation of CaOx nanocrystal precipitates within the enamel lamella and dentin tubule complex provide effective immediate chemical chelation with the HAp of enamel, especially the dense peritubular wall of the dentin tubules in the middle and more pulpal zones of tubules. By achieving a rapid comprehensive chelation in the broader surface areas of multiple zones along the EDJ as well as within the dentin tubules, an exponential-non-linear advantage accumulates to prevent patient sensitivity. This is due to the increased inner diameter of the tubules from the outermost EDJ interfacial zone to the innermost dentin pulp zones, with the largest tubule diameter that exists being at the pulp interface. Without the composition enabling chelation to the mineral surfaces, these increasingly larger peritubular surfaces would not provide architecture for a mechanical blockage of the tubule inner diameter. The results are that these open tubule regions remain as likely zones of bidirectional fluid flow, which causes pain or discomfort, and increased tooth sensitivity.

One advantage of the present composition is that it can induce the formation of CaOx in the oral cavity within the dentinal tubule complex to limit or prevent the entry of microorganisms into the enamel and dentin tubule complex. In some embodiments, the composition does not interfere with the application and interdiffusion of any hydrophilic adhesive primer system that supports the formation of a hybrid layer into the subjacent inter-tubular and tubular dentin. Various aerobic and anaerobic bacteria have an affinity for the dentin tubule luminal walls, with a particular attraction that is associated with streptococcal species. Contents of dentin tubules may contain type-I collagen, which is recognized by oral streptococci and serves as an adhesive substrate when absorbed onto the HAp crystals of tooth substrates. For example, strains of S. mutans can bind to un-mineralized collagen and root dentin surfaces. Consequently, the binding of oral streptococci to collagen may facilitate bacterial adhesion to exposed dentin or cementum and subsequently enable tubule invasion leading to caries. By blocking the tubule, the present composition can limit or prevent bacterial invasion and the formation of caries.

Indeed, streptococcal protein adhesins can interact with salivary molecules and favor the colonization by a range of mechanisms in the oral cavity. Bacterial aggregation and colony formation is an important aspect in plaque development. The streptococci can co-adhere with other bacterial colonizers, such as Actinomyces, P. gingivalis and Bacteroides forsythus into a dense microbiome. A polypeptide antigens expressed on the surface of most species of oral streptococci, is involved in mediating adhesion of streptococci to collagen on the inner walls of dentin tubules thereby facilitating bacterial adhesion and invasion of dentin tubules. The present composition induces the chelation reaction to block the dentin tubules thereby denying bacterial access to the associated collagen thus reducing the natural colonization risk of streptococci and other oral microorganisms. The nanometric size of the oxalic acid potassium salt dihydrate crystals improves the blockage of collagen by having an increased density and a tighter blockage of the dentin tubule when compared, for example, to micron sized potassium salt crystals.

A sufficient concentration of oxalic acid potassium salt dihydrate crystals should be included in the composition in order to create a sufficient density of crystals in the oral cavity (e.g. inside the dentin tubules) to obtain the benefits of the composition. A high density enables sufficient residence time of the oxalic acid potassium salt dihydrate crystals adjacent to lamellae and tubules to promote deep penetration and increased chelation or occlusion of these voids or channels through diffusion. A concentration of 1.5 to 15 wt. %, 1.5 to 10 wt. %, 1.5 to 8 wt. %, 1.5 to 7 wt. %, 1.5 to 5 wt. % or preferably 1.5 to 4 wt. % of oxalic acid potassium salt dihydrate crystals was found to provide the pain relief and teeth desensitization effect described, as well as a sufficient density of oxalic acid potassium salt dihydrate crystals in the oral cavity. The oxalic acid potassium salt dihydrate crystals can have a size of from 50 nm to less than 1000 nm, from 50 to 900 nm, from 50 to 800 nm, from 50 to 750 nm, from 90 to 1000 nm, from 100 to 1000 nm, from 100 to 900 nm, from 100 to 800 nm or from 100 to 700 nm.

The present composition comprises from 0.5 to 10 g/L of NaOH which plays a role in stabilizing the oxalic acid potassium salt dihydrate crystals to achieve a size in the nanometric range of less than 1000 nm. NaOH can be provided in a concentration range of 0.5 to 10 g/L, 0.75 to 10 g/L, 0.5 to 7.5 g/L, 0.5 to 5 g/L or 1 to 5 g/L. Based on Le Chatelier's equilibrium law, the density of oxalic acid potassium salt dihydrate crystals can be increased by the addition of NaOH in the described concentration ranges. The chemistry of the equilibrium reaction drives the final solubility to readjust itself to counteract the stoichiometric change and establish a new system symmetry, which increases the final concentration of oxalic acid potassium salt dihydrate crystals. In other words, the addition of NaOH increases the solubility of oxalic acid potassium salt dihydrate crystals which in turn improves the chelation reaction with the HAp of the enamel, dentin and cementum to produce CaOx. The CaOx can have a size similar to that of the oxalic acid potassium salt dihydrate crystals, for example from 100 nm to more than 1000 nm.

The presence of NaOH in these concentration ranges creates an altered chemical equilibrium which increases the density of the oxalic acid potassium salt dihydrate crystals as well as the resulting acid resistant CaOx nanocrystals after chelation. The increase in solubility of oxalic acid potassium salt dihydrate crystals promotes a resulting nanometric size of the crystals. In some embodiments, a mass ratio of oxalic acid potassium salt dihydrate crystals to NaOH is from 5:1 to 15:1, from 7:1 to 13:1, from 8:1 to 12:1, or can be 10:1. The relatively smaller content of sodium hydroxide in the solution compared to potassium salt allows to maintain a pH of less than 2.0 while obtaining the benefit of the NaOH base for stabilizing the nanometric size of the crystals.

In optional embodiments, a fluoride can be added to the composition to provide antimicrobial activity, promote the formation of fluorapatite, and help stabilize the acidic pH to a value as described herein such as below 2. Fluoride may be incorporated into the composition through the use of various fluoride ion sources, such as sodium fluoride, amine fluoride, sodium monofluorophosphate (MFP), stannous fluoride, and potassium fluorostannite. Fluoride can prevent caries, especially in children and adults who are at high risk for caries attack rates that can be observed as white spot lesions. Fluoride can promote the remineralization of white spot lesions, to inhibit the progression of caries. The addition of fluoride can reduce or halt the white-spot demineralization through the cervical enamel pores which are developmental remnants of a hypomineralized enamel substrate that are found along the enamel-gingival margin as well as initiating the remineralization of the subjacent amorphous pore area below the same white spot enamel lesions.

Generally, when provided in an oral composition, the concentration of fluoride in dental enamel and HAp substrates is greatest on the surface and lessens as the subjacent substrate is penetrated. Fluoride combines physically and chemically with the HAp of the tooth, which decreases the solubility of organic acids in bacterial biofilms. In addition, fluoride reduces the growth of plaque forming microorganisms on tooth surfaces by altering bacterial enzymes that hydrolyze the carbohydrates into organic acids. By inhibiting the growth of microorganisms (i.e. a germicidal effect), their release of organic acids is thus reduce. The organic acids can cause demineralization of enamel and dentin substrates which results in the formation of new and recurrent caries.

Accordingly, fluoride agents promote long term germicidal action against microorganisms that cause caries, gingivitis, periodontitis, and peri-implantitis. Fluoride can inhibit tooth demineralization of subsurface enamel white spot lesion HAp crystals that lie below the amorphous pores and outer enamel surface along the thin cervical one third of enamel-gingival interface in the human teeth. The same fluoride ion that inhibits demineralization can simultaneously promotes remineralization of the calcium subsurface HAp crystals, which lie below the white post enamel surface. These combined effects can halt the carious process to prevent the cavitation (breaking) of the amorphous enamel surface layer that often fractures, causing a carious lesion that requires clinical operative intervention.

In the chelation reaction, the substitution of the OH⁻ with a fluoride ion can change the HAp to a fluorapatite, which is more acid resistant to the organic acids of the caries created by microorganisms. The pH chemistry of the present composition being less than 2 improves the chelation with the HAp of tooth and promotes the formation of a dense precipitate of CaOx-fluorapatite nanocrystals that fill the enamel lamella and defects as well as the interfacial EDJ in the dentin tubule complex. With the addition of fluoride in the composition, oxalic acid potassium salt dihydrate crystals can be chelated to a durable long-lasting bacteriometic seal that includes fluorapatite.

Another advantage of the optional addition of fluoride is that the fluoride enhances the potassium salt chemistry and helps stabilize the pH of the solution to below 2.0. The fluoride complexes with the HAp to form a dense crystal complex of CaOx fluorapatite nanocrystals that are resistant to the organic acids of the carious microorganisms.

To achieve the advantages described above, fluoride can be optionally provided in the composition in a concentration of 100 ppm to 1500 ppm, preferably 250 ppm to 1000 ppm. In other embodiments, the concentration of fluoride in the composition is from 0.01 to 0.5 μg/L, 0.05 to 0.25 μg/L or 0.1 μg/L. In further embodiments, fluoride ion is provided in the concentration range of 0.020 to 0.25 wt. % and more preferably from 0.3 to 0.34 wt. %.

In some embodiments, the composition optionally further includes a high oxygen potential (HOP) water to enhance the germicidal effect to neutralize pathogenic microorganisms which cause acute and chronic caries, gingivitis (which often leads to periodontitis, which itself often leads to alveolar bone loss).

The HOP water can inhibit the initial aggregation of microorganisms that cause caries and can reduce or prevent the invasion of microorganisms that cause gingivitis, periodontitis and alveolar bone loss.

HOP water can generated by electrolysis that produces a oxygen-reduction potential (redox potential) of more than 11 mV at a pH of 2.0 or less. At a neutral pH 7.0, most natural untreated water contains equal concentrations of H⁺ and OH⁻. However, the HOP super-oxidizing acidic water has low pH values and contains high concentrations of H⁺ ions. An HOP water with a redox potential of 1100 mV or more is a superoxidizing acidic agent that withdraws electrons from microorganisms (e.g. bacteria fungi and molds). Moreover, the HOP water is non-toxic in the oral cavity and presents no environmental risk.

The advantages of optionally including HOP water are several. Firstly, the use of HOP water with a pH of 2.0 or less improves the long-term pH stability of the composition. Second, the addition of the HOP water provides the germicidal effect described above. In combination with fluoride, this further ensures that bacteria are neutralized and are not able to penetrate through the enamel defects and dentin tubules when the present composition is administered to an oral cavity. When the HOP water is provided in the composition, it may be present in concentration range of from 0.1 mL/L to 10 mL/L, 0.25 mL/L to 7.5 mL/L, 0.5 to 5 mL/L, 0.5 to 2 mL/L or 1 mL/L.

The composition can optionally further include one or more of a flavoring agent, a coloring agent, or an antiseptic agent. In one example, the antiseptic agent is a bisbiguanide such as alexidine and/or chlorhexidine. The coloring agent may be a dye that produces an agreeable patient friendly color. The flavoring agent can be of several natural or non natural sweeteners (e.g. stevia) to promote a pleasing taste to the subject receiving the composition. Common flavors include mint and various fruit flavors such as strawberry, apple, and the like.

The composition can be formulated as an aqueous composition (e.g. mouth rinse), a tooth gel (e.g. pediatric tooth gel), a toothpaste, prophy paste, a cavity liner, an amalgam, an adhesive composite, a glass ionomer, gold foil restorations, or extracoronal inlays, onlays and crowns. The composition may also be incorporated into a existing compositions of dentifrice paste, gel or polishing agent such as a dicalcium phosphate, resinous polishing agents or gelling components that are useful for brushing of sensitive teeth. The gelling agent may be a carrageenan or xanthan gum and may be incorporated with a polishing agent such as silica or a carbapol. In addition, the components of the invention may be made in a liquid medium to make a toothpaste, mouthrinse, a chewing gum or a prophy paste.

The composition can also be formulated or incorporated into over the counter (OTC) dental formulations that provide pain relief for both human and mammals such as dogs, cats, ferrets, and horses (e.g. racing horses). The present composition can provide pain relief and tooth desensitization in animals having occlusal rasping of constantly erupting molars. This condition is common in animals in the livestock industry who suffer from tooth pain due to the continual eruption of their posterior chewing teeth.

The composition described herein can be produced by mixing an oxalic acid potassium salt and a NaOH salt in an aqueous phase (for example purified water). The amounts of oxalic acid potassium salt and NaOH salt are such that the final concentration ranges of NaOH and oxalic acid potassium dihydrate salt is as described above. In preferred embodiments, the oxalic acid potassium salt is a medical grade salt of high purity, e.g. 99% pure or more. The mixture obtained is then heated to a temperature above room temperature. The mixture can be further subjected to additional mixing while the heat is applied. In some embodiments, the temperature is increased until the mixture becomes clear to the naked eye. For example, the temperature can be from 28 to 40° C., from 28 to 35° C. or from 29 to 31° C. and the heating may be applied for 30 mins-1 h30. The present composition can therefore be considered a supersaturated solution since the composition is heated to increase the solubility of the salts and is then cooled while retaining the dissolved salts. After the heating, the pH of the composition is adjusted to be less than 2.0 by adding an acid or an acidic composition (e.g. HOP water). In some embodiments, the pH adjustment includes adding HOP water having a pH of from 1 to 1.9. The suspension obtained after pH adjustment is then subjected to an ultrasonication until the oxalic acid potassium dihydrate salt crystals have a size of less than 1000 nm. In one example, the suspension may be ultrasonicated in a closed fluid circuit. The addition of fluoride can be optionally performed before and/or after the ultrasonication. For example, the addition of fluoride can be done in increments.

The present composition can be used in the treatment of oral pain, teeth sensitivity, caries, orthodontic banding and/or as a bacteriometic seal for use in periodontal scaling and root planning. After an injury, surgery or other extreme sudden stimuli, oral pain and tooth sensitivity can develop and may be treated with the composition. For example, tooth sensitivity may occur as the result of postoperative hypersensitivity following the initial restorative placement of an existing amalgam alloy, a composite restoration or the replacement of a failed restoration following the bleaching of teeth with light, heat, or other power assisted forms of tooth whitening, or for many other reasons where tooth enamel is damaged and dentin becomes exposed.

The product of the present invention can be applied on prepared tooth structure such vital dentin both before and after oral hygiene treatment for prophylaxis for cleaning and scaling. The product may be used as a one-step replacement under crowns and inlays with veneer preparation. It can be used on the dentin of all cavity preparation for amalgam alloys, and resin composite restoration. The acid resistant film forming liner material can have bonding materials applied directly on its surface for binding restorative materials. It may also be applied on the tooth surface following a bleaching procedure whether the procedure is done in a dentist's office or if the subject uses a home bleaching kit. In addition, the composition can be used as a diagnostic tool to differentiate between acute dentinal pain and chronic pulpal pain. Acute dentin pain is generally called a reversible tooth pain. To the dentist and subject, this means that there is a defect located within the substance of the dentin and not within the nerves within the dental pulp. The problem is reversible without any invasive endodontic treatment. Alternatively, chronic dental pain is an irreversible stimulus which indicates that the nerves of the dental pulpal are inflamed and must be removed by some sort of biomechanical endodontic instrumentation. The present composition provides a simple one-step diagnostic treatment that allows the dentist to discriminate reversible and irreversible dental pain. When a patient complains of pain to cold and air and there are no diagnostic features of radiographic presence of a periapical radiolucency, fractured tooth root or other obvious clinical problems then the dentist may simply rub the composition onto and around edges or cavosurface margins of the tooth restoration interface. If the patient reports an immediate cessation to dental pain then the dentist may complete the diagnosis that the problem is fluid flow in the dentin or microleakage. This is confirmation of reversible pulp inflammation and may be treated by the repair of the restoration and not the removal of the pulp.

### Example

A general exemplary protocol for producing the composition is described. 30 g of oxalic acid potassium salt with 99% purity was added to 1 L of purified water or an electrolytically treated purified water in a mixing container. 1 g of NaOH was added to the water and mixed. The mixture was heated to about 29° C. The container was covered to prevent entrance of foreign agents and placed into an ultrasonic water bath. The ultrasonic bath was started at a setting of constant duty cycle, time on hold, output control at 0.9 or 18,000 Hz. The solution was then subjected to ultrasonic energy dissociation for around 60 minutes. Fluoride was then added (0.1 μg) while the mixture was still heated. The mixture was observed to be sure that it became soluble in the final mixture during sonication. Samples were taken for viewing with a 100× scanning electron microscope (SEM). The pH of the final solution was between 1.02 and 1.85.

Other compositions were produced according to the above protocol (but without fluoride and without HOP) and were labeled compositions A-G. Compositions A-G had variable contents of oxalic acid potassium salt and NaOH salt. The compositions are presented in Table 1 below.

TABLE 1
Compositions A-G and controls

| | oxalic acid potassium salt | Sodium hydroxide |
|---|---|---|
| Composition | (in g) | (in g) |
| A | 40.0 | 4 |
| B | 29.9 | 1 |
| C | 28.6 | 1 |
| D | 26.6 | 1 |
| E | 24.0 | 1 |
| F | 22.0 | 1 |
| G | 20.0 | 1 |
| Control 1 | 29 | 0 |
| Control 2 | 24.0 | 0 |

A total of 15-human non-carious teeth were extracted for orthodontic purposes under and maintained in a moist state of a 1% sodium azide solution that is germicidal to microorganisms found in the oral cavity. The oral surfaces of 5-teeth were observed at 20X under a stereomicroscope and evaluated for location of enamel lamella and other defects by drying the surface with a jet of air for several seconds. The lamella and defects were identified, by showing a white cleft that projected into the substance of the enamel substrate. The entire area-including the lamella and defects-were treated by rubbing the entire enamel surface with a microbrush that was saturated with composition A for 5 seconds and then, gently, air was dispersed with an air syringe for 1 second bursts at a distance of 10 cm from the teeth.

Each tooth was mechanically split leaving a fractured en-face interface of the enamel-dentin interface for SEM observation. The fractured enamel surfaces were then prepared for observation by SEM technical procedures. It was observed that all of the enamel lamellae and defects as well as those areas of the EDJ and subjacent dentin tubules beneath the EDJ of the enamel lamella contained chelated CaOx nano-crystals that had filled the defects. Many of the lamella and defects showed areas in which the CaOx nano-crystals were lost due to the mechanical fracturing process that had been performed prior to evaluation by SEM.

An additional 10-teeth, which had been extracted for orthodontic purposes were removed from the azide solution and stabilized in a holding block whereby each tooth received a class-V facial/buccal cavity from mesial to distal corners on each tooth with a new diamond bur. The cavities were immediately treated for a period of 5-secs with a microbrush that had been saturated with the composition A. The teeth were then fractured in a manner to provide an en face view of the cavity floor and walls interface.

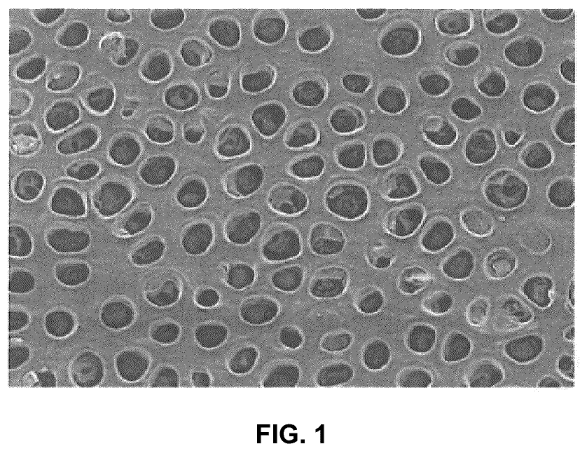

FIG. 1 is a 2,000×SEM image of the dentin floor that was acid etched to remove the operative smear grinding debris with a rotary bur. The dark holes are open dentin tubules from the etching. It can be observed that some contain organic proteins, fluid and nerve fibers. The perimeter of each tubule shows a white zone of dense peritubular dentin. It was estimated that the intertubular dentin contained around 30% of organics.

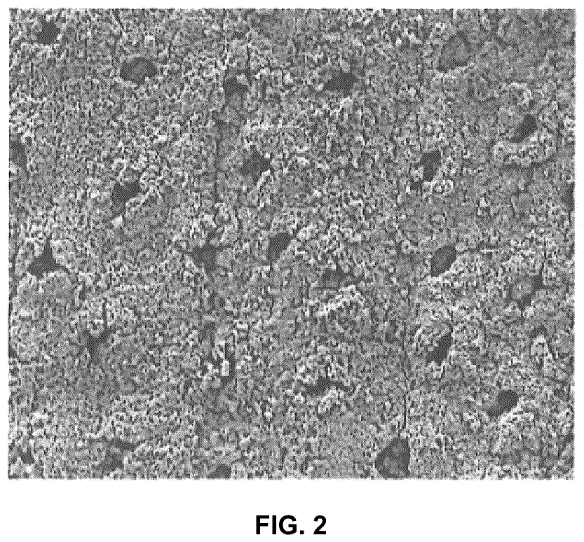

FIG. 2 shows a 2,000×SEM image of the dentin floor that was acid etched to remove the operative smear debris and treated for 5 seconds with composition A. It can be seen that the entire cavity floor is covered with many millions of small multiform CaOx nanocrystals having a size ranging from 100 nm to 700 nm. Each dentin tubule was observed to have been filled with CaOx nanocrystals that are thus blocking the fluid flow across the tubule which causes teeth sensitivity.

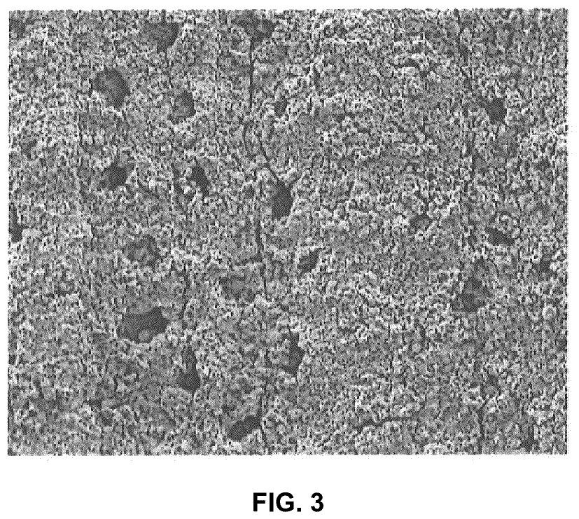

FIG. 3 shows a 5,000×SEM image of the dentin floor that was acid etched to remove the operative smear debris and then treated for 5 seconds with composition A. The entire cavity floor was covered with many millions of small multiform CaOx nanocrystals having a size ranging from 100 nm to less than 1000 nm. Each dentin tubule was observed to have been filled with CaOx nanocrystals.

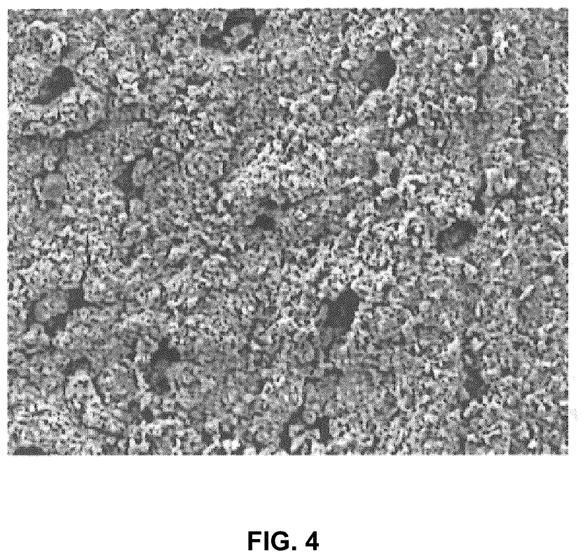

FIG. 4 shows a 7,000×SEM image of the dentin floor that was acid etched to remove the operative smear debris and then treated for 5 seconds with composition A. The entire cavity floor was covered with many millions of small multiform CaOx nanocrystals having a size ranging from 100 nm to less than 1000 nm. Each dentin tubule was observed to have been filled with CaOx nanocrystals.

Several drops of each compositions A-G identified below were placed onto trays with a HAp mineral substrate. All of compositions A-G precipitated into nanocrystals of CaOx. It was noted that the nanocrystals were smaller for compositions A-G when compared to Control 1.

To further evaluate the solubility of the oxalic acid potassium salt with NaOH, 5 samples of “control 2” and 5 samples of composition E were placed in sealed containers and stored for at least 168-hours at 15.0° C. Each sample was evaluated at a 24-hour interval and photographed. The results demonstrated that composition E consistently reached full dissolution much faster than the “control 2” samples. The average difference was 173.6-hours, meaning that composition E reached its solubility endpoint 7-days earlier than the control. These results demonstrate that the inclusion of the NaOH in a solution of oxalic acid potassium salt effectively increased the rate of solubility. The addition of the NaOH increased the concentration of water (i.e. OH⁻ reacted to form more water H₂O). NaOH was demonstrated to reduce level of ions in the aqueous phase (i.e. ion equilibrium shift) and add alkalinity. The shift in equilibrium also increases the solubility of the oxalic acid potassium salt in water.

With the addition of 2 moles of NaOH to an oxalic acid potassium salt solution, there are 2 moles of water produced and 1 mole of NaOx formed, meaning that 1 mole of the potassium is broken down into 1 mole of NaOx. In other words, for every 2 moles of NaOH there are 1 mole of KOx that is removed (i.e. the concentration is reduced by 1 mole). The change in the acid-base equilibrium affects solubility by shifting the equilibrium towards an ionized state and drives the potassium-salt into solution. SEM evaluation of increased multiform CaOx morphologies provided visual confirmation, that solutions of potassium-salt with NaOH occludes the cavity dentin surface floor of prepared human dentin cavity floor tubules by forming CaOx nano-crystals.

What is claimed is:

1. A composition for teeth desensitization comprising:
from 1 to 15 wt. % of suspended crystals of oxalic acid potassium salt dihydrate having a size of from 50 nm to less than 1000 nm;
from 0.5 to 10 g/L of NaOH; and
wherein the composition has a pH of less than 2.0.

2. The composition of claim 1, wherein the suspended crystals of oxalic acid potassium salt are present in a concentration of from 1.5 to 7 wt. %.

3. The composition of claim 2, wherein the concentration is of from 1.5 to 4 wt. %.

4. The composition of claim 1, wherein the size the suspended crystals of oxalic acid potassium salt is of from 100 to 800 nm.

5. The composition of claim 1, wherein the NaOH is present in a concentration of from 1 to 5 g/L.

6. The composition of claim 1, wherein a mass ratio of the oxalic acid potassium salt dihydrate crystals to the NaOH is from 5:1 to 15:1.

7. The composition of claim 6, wherein the ratio is from 8:1 to 12:1.

8. The composition of claim 1, wherein the pH is between 1 and 2.

9. The composition of claim 1, wherein the pH is between 1 and 1.9.

10. The composition of claim 1, further comprising fluoride.

11. The composition of claim 10, wherein the fluoride is present in a concentration of 100 ppm to 1500 ppm.

12. The composition of claim 1, further comprising high oxygen potential water.

13. The composition of claim 12, wherein the high oxygen potential water is present in a concentration of from 0.1 mL/L to 10 mL/L.

14. The composition of claim 1, further comprising a flavoring agent, a coloring agent, or an antiseptic agent.

15. The composition of claim 14, wherein the antiseptic agent is a bisbiguanide.

16. A method for producing a composition for teeth desensitization, the method comprising:
mixing an oxalic acid potassium salt and a NaOH salt in an aqueous phase to obtain a mixture;
heating the mixture to a temperature of from 28 to 40° C.;
reducing the pH of the mixture to less than 2.0 to obtain an acidic mixture; and
ultrasonicating the acidic mixture to obtain oxalic acid potassium dihydrate salt crystals having a size of less than 1000 nm.

17. The method of claim 16, wherein the step of reducing the pH includes adding high oxygen potential water.

18. The method of claim 16, wherein the step of ultrasonicating comprises ultrasonicating in a closed fluid circuit.

19. The method of claim 16, wherein the step of heating is performed for 30 mins-1 h30.

20. The method of claim 16, further comprising, after the step of reducing the pH and/or after the step of ultrasonicating, adding a fluoride.
